(19) United States
(12) Patent Application Publication
(10) Pub. No.: US 2008/0027371 A1
(43) Pub. Date: Jan. 31, 2008

(54) Method and device for minimally invasive site specific ocular drug delivery

(76) Inventors: John W. Higuchi, Salt Lake City, UT (US); Anthony L. Tuitupou, Salt Lake City, UT (US)

Correspondence Address: THORPE NORTH & WESTERN, LLP., 8180 SOUTH 700 EAST, SUITE 350, SANDY, UT, 84070, US

(21) Appl. No.: 11/881,729

(22) Filed: Jul. 26, 2007

Related U.S. Application Data
(60) Provisional application No. 60/820,461, filed on Jul. 26, 2006.

(51) Int. Cl. A61N 1/30; A61F 2/00; A61K 31/44; A61K 31/56

(52) U.S. Cl. 604/20; 424/427; 514/169; 514/172; 514/282

(57) ABSTRACT

The present invention includes techniques for delivering an active agent into the eye of a subject. Accordingly, in one aspect a method may include delivering invasively an active agent into a peripheral tissue of the eye to form a drug reservoir, and applying an electric current to the drug reservoir to thus drive at least a portion of the active agent at least partially through the choroid. Numerous configurations are contemplated for the positioning of the electric current relative to the drug reservoir. For example, in one aspect the electric current may be applied to the drug reservoir from a non-invasively positioned electrode. In another aspect, the electric current may be applied to the drug reservoir from an invasively positioned electrode. A variety of invasive positions are contemplated, including, for example, positioning the invasive electrode within the peripheral tissue. In yet another aspect, delivering the active agent may further include implanting an invasive electrode having an associated drug reservoir containing the active agent into the peripheral tissue.

### PRIORITY DATA

This application claims the benefit of U.S. Provisional Patent Application Ser. No. 60/820,461, filed on Jul. 26, 2006, which is incorporated herein by reference.

### FIELD OF THE INVENTION

The present invention relates to the delivery of an active agent through a localized region of a subject's eye. Accordingly, the present invention involves the fields of chemistry, pharmaceutical sciences, and medicine, particularly ophthalmology.

### BACKGROUND OF THE INVENTION

Posterior and intermediate eye diseases that require ocular drug delivery to prevent blindness include uveitis, bacterial and fungal endophthalmitis, age-related macular degeneration, viral retinitis, and diabetic retinopathy, among others. For example, the reported incidence of posterior uveitis is more than 100,000 people in the United States. If left untreated, uveitis leads to blindness. It is responsible for about 10 percent of all visual impairment in the U.S. and is the third leading cause of blindness worldwide.

Treatments of intermediate and posterior uveitis are complicated by the inaccessibility of the posterior eye to topically applied medications. Current therapy for intermediate and posterior uveitis requires repeated periocular injections and/or high-dose systemic therapy with corticosteroids. Injections are usually preferred to systemic drug administration because the blood/retinal barrier impedes the passage of most drugs from the systemically circulating blood to the interior of the eye. Therefore large systemic doses are needed to treat intermediate and posterior uveitis, which often result in systemic toxicities including immunosuppression, adrenal suppression, ulcerogenesis, fluid and electrolyte imbalances, fat redistribution and psychological disorders.

As another example, endophthalmitis affects approximately 10,000 people in the United States each year. Endophthalmitis is typically caused by gram-positive bacteria after ocular surgery or trauma, but it can also be fungal or viral in nature. The current method of treating endophthalmitis is direct injection of antimicrobials into the vitreous. Intravitreal injections are necessary because periocular injections and systemic administration do not deliver efficacious amounts of antibiotics to the target sites in the eye. Additionally, age-related macular degeneration (AMD) is the leading cause of irreversible loss of central vision in patients over the age of 50. AMD affects more than 15 million people worldwide.

Treatments of posterior eye diseases require intravitreal and periocular injections or systemic drug administration. Systemic administration is usually not preferred because of the resulting systemic toxicity as discussed above. While intravitreal and periocular injections are preferable to systemic administration, the half-life of most injected compounds in the vitreous is relatively short, usually on the scale of just a few hours. Therefore, intravitreal injections require frequent administration. The repeated injections can cause pain, discomfort, intraocular pressure increases, intraocular bleeding, increased chances for infection, and the possibility of retinal detachment. One major complication of periocular injections is accidental perforation of the globe, which causes pain, retinal detachment, ocular hypertension, and intraocular hemorrhage. Other possible complications of periocular injections include pain, central retinal artery/vein occlusion, and intraocular pressure increases. Therefore, these methods of ocular drug delivery into the posterior of the eye have significant limitations and major drawbacks.

Ocular iontophoresis is a noninvasive technique used to deliver compounds of interest into the interior of a patient's eye. In practice, two iontophoretic electrodes are used in order to complete an electrical circuit. In traditional, transscleral iontophoresis, at least one of the electrodes is considered to be an active iontophoretic electrode, while the other may be considered as a return, inactive, or indifferent electrode. The active electrode is typically placed on an eye surface, and the return electrode is typically placed remote from the eye, for example on the earlobe. The compound of interest is transported at the active electrode across the tissue when a current is applied to the electrodes. Compound transport may occur as a result of a direct electrical field effect (e.g., electrophoresis), an indirect electrical field effect (e.g., electroosmosis), electrically induced pore or transport pathway formation (electroporation), or a combination of any of the foregoing. Examples of currently known iontophoretic devices and methods for ocular drug delivery may be found in U.S. Pat. Nos. 6,319,240; 6,539,251; 6,579,276; 6,697,668, and PCT Publication Nos. WO 03/030989 and WO 03/043689, each of which is incorporated herein by reference.

One potential problem with present ocular iontophoretic methods and devices concerns the actual delivery, or rather, the non-delivery of the drug into the eye tissue. Because the return electrode is located remote from the eye, various conductive pathways may be formed. Such divergence of the electric current will decrease the efficiency of drug delivery to the target sites in the eye, and as a result, much of the drug may be delivered into the tissues surrounding the eye rather than into the eye per se. Furthermore, delivery of a drug to posterior ocular tissues can be challenging due to the difficulty in applying current to such tissues.

As such, devices, systems, and methods which are capable of delivering a drug to the eye in a therapeutically effective manner, particularly to the posterior of the eye, continue to be sought.

### SUMMARY OF THE INVENTION

Accordingly, the present invention provides a method of delivering an active agent into an eye of a subject. Such a method may include delivering invasively an active agent into a peripheral tissue of the eye to form a drug reservoir, and applying an electric current to the drug reservoir to thus drive at least a portion of the active agent at least partially through the choroid. Numerous configurations are contemplated for the positioning of the electric current relative to the drug reservoir. For example, in one aspect the electric current may be applied to the drug reservoir from a non-invasively positioned electrode. In another aspect, the electric current may be applied to the drug reservoir from an invasively positioned electrode. A variety of invasive positions are contemplated, including, for example, positioning the invasive electrode within the peripheral tissue. In yet another aspect, delivering the active agent may further include implanting an invasive electrode having an associated drug reservoir containing the active agent into the peripheral tissue.

Iontophoretic delivery may be facilitated by using a return electrode to complete an electric circuit within the eye of the subject. In one aspect, for example, an electrical circuit may be completed with the invasive electrode via a return electrode positioned within the peripheral tissue. In addition to completing an electric circuit, in some aspects the return electrode may be utilized to iontophoretically deliver a secondary agent from an associated secondary reservoir into the eye. Although a variety of secondary agents are contemplated, in one aspect such an agent may include a depot forming agent. In addition to invasive return electrodes, the present invention also provides aspects utilizing non-invasive return electrodes. Such electrodes may be positioned on a surface of the eye, or they may be remote from the eye on a structure such as an earlobe.

Numerous active agents are contemplated for incorporation in the drug reservoir, all of which are considered to be within the present scope. In one aspect, for example, the drug reservoir may include an active agent selected from hydromorphone, dexamethasone, dexamethasone phosphate, amikacin, oligonucleotides, F_{ab} peptides, PEG-oligonucleotides, salicylate, tropicamide, methotrexate, 5-fluorouracil, squalamine, triamcinolone acetonide, triamcinolone acetonide phosphate, diclofenac, combretastatin A4, mycophenolate mofetil, mycophenolic acid, bevacizumab, ranibizumab, and prodrugs and combinations thereof. In one specific aspect, the active agent may be triamcinolone acetonide phosphate. In another specific aspect, the active agent may be dexamethasone phosphate. It should be noted that the active agent delivered into the eye may provide immediate therapeutic effect, sustained therapeutic effect, or both immediate and sustained therapeutic effect.

As has been suggested, in some aspects a secondary agent may be delivered to the eye of the subject. In one aspect, for example, the secondary agent may be invasively delivered with the drug reservoir. In another aspect, the secondary agent may be non-invasively delivered with the electric current.

A variety of secondary agents are contemplated, including depot forming agents, active agents, vasoconstrictor agents, solubility modifying agents, and combinations thereof. In one aspect, for example, the secondary agent may be a vasoconstrictor agent. Non-limiting examples of vasoconstrictor agents may include naphazoline, tetrahydrozoline, phenylethylamine, epinephrine, norepinephrine, dopamine, dobutamine, colterol, ethylnorepinephrine, isoproterenol, isoetharine, metaproterenol, terbutaline, metearaminol, phenylephrine, tyramine, hydroxyamphetamine, ritrodrine, prenalterol, methoxyamine, oxymetazoline, albuterol, amphetamine, methamphetamine, benzphetamine, ephedrine, phenylpropanolamine, methentermine, phentermine, fenfluramine, propylhexedrine, diethylpropion, phenmetrazine, phendimetrazine, and combinations thereof. In one specific aspect, the vasoconstrictor agent may be oxymetazoline.

### BRIEF DESCRIPTION OF THE DRAWINGS

FIG. 1 is a section view in accordance with an aspect of the present invention.

FIG. 2 is a section view in accordance with another aspect of the present invention.

FIG. 3 is a section view of a delivery instrument in accordance with yet another aspect of the present invention.

FIG. 4 is a section view in accordance with a further aspect of the present invention.

FIG. 5 is a section view in accordance with another aspect of the present invention.

### DETAILED DESCRIPTION OF THE INVENTION

Before the present systems and methods for ocular drug delivery are disclosed and described, it is to be understood that this invention is not limited to the particular process steps and materials disclosed herein, but is extended to equivalents thereof, as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and, “the” include plural referents unless the context clearly dictates otherwise. Thus, for example, reference to “a polymer” includes reference to one or more of such polymers, and “an agent” includes reference to one or more of such agents.

Definitions

In describing and claiming the present invention, the following terminology will be used in accordance with the definitions set forth below.

As used herein, “formulation” and “composition” may be used interchangeably herein, and refer to a combination of two or more elements, or substances. In some embodiments a composition may include an active agent, an excipient, or a carrier to enhance delivery or depot formation.

As used herein, “peripheral tissue” refers to tissues and/or spaces between tissues that are located between the conjunctiva and the choroid. Examples of such tissues and/or spaces between tissues may include subconjunctival, episcleral, intrascleral, deep scleral, suprachoroidal space, etc.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts. Examples of drugs useful in the present invention include without limitation, steroids, antibacterials, antivirals, antifungals, antiprotozoals, antimetabolites, immunosuppressive agents, VEGF inhibitors, ICAM inhibitors, antibodies, protein kinase C inhibitors, chemotherapeutic agents, neuroprotective agents, nucleic acid derivatives, aptamers, proteins, enzymes, peptides, and polypeptides.

As used herein “prodrug” refers to a molecule that will convert into a drug (its commonly known pharmacological active form). Prodrugs themselves can also be pharmacologically active, and therefore are also expressly included within the definition of an “active agent” as recited above. For example, dexamethasone phosphate can be classified as a prodrug of dexamethasone, and triamcinolone acetonide phosphate can be classified as a prodrug of triamcinolone acetonide.

As used herein, “effective amount,” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “sclera” refers to the sclera tissue in the eye or the conjunctiva between the limbus and the fomix on the surface of the eye, which is the white part of the eye. “Sclera” is also used in referring to other eye tissues.

As used herein, “subject” refers to a mammal that may benefit from the administration of a composition or method as recited herein. Examples of subjects include humans, and may also include other animals such as horses, pigs, cattle, dogs, cats, rabbits, and aquatic mammals.

As used herein, “administration,” and “administering” refer to the manner in which an active agent, or composition containing such, is presented to a subject. As discussed herein, the present invention is primarily concerned with ocular delivery.

As used herein, “noninvasive” refers to a form of administration that does not rupture or puncture a biological membrane or structure with a mechanical means across which a drug or compound of interest is being delivered. A number of noninvasive delivery mechanisms are well recognized in the transdermal arts such as patches and topical formulations. Many of such formulations may employ a chemical penetration enhancer in order to facilitate non-invasive delivery of the active agent. Additionally, other systems or devices that utilize a non-chemical mechanism for enhancing drug penetration, such as iontophoretic devices are also known. “Invasive” refers to a form of administration that punctures a biological membrane or structure.

As used herein, “depot” or “drug depot” refers to a temporary mass inside a biological tissue or system, which includes a drug that is released from the mass over a period of time. In some aspects, a depot may be formed by the interaction of an active agent with a depot forming agent, such as a complexing ion which will form an active agent complex that is less soluble than the active agent by itself, and thus precipitate in-vivo.

As used herein, the term “surface” with respect to the eye refers to an outer tissue surface of the eye that is encountered in ocular delivery.

As used herein, the term “reservoir” refers to a body or a mass that may contain an active agent, a secondary compound, or other pharmaceutically useful compound or composition. As such, a reservoir may include any structure that may contain a liquid, a gelatin, a semi-solid, a solid or any other form of active agent or secondary compound known to one of ordinary skill in the art. In some cases, an electrode may be considered to be a reservoir.

As used herein, the term “active electrode” refers to an electrode utilized to iontophoretically deliver an active agent.

As used herein, the term “passive electrode” refers to an electrode that is used to complete an electrical circuit without delivering a compound or substance to a subject.

As used herein, the term “return electrode” refers to an electrode utilized to complete an electrical circuit for active electrode. In one aspect, a return electrode may be an active electrode used to deliver a secondary compound, such as an active agent, a depot forming agent, etc. In another aspect, a return electrode may be a passive electrode.

As used herein, the term “reacting” refers to any force, change in environmental conditions, presence or encounter of other chemical agent, etc. that alters the active agent. For example, “reacting” between the active agent and the depot forming agent can be physical or chemical interactions.

As used herein, the term “precipitate” refers to anything less than fully solubilized. As such, a precipitate can include not only crystals, but also gels, semi-solids, increased molecular weight, etc.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

The Invention

The present invention provides methods for delivering an active agent into the eye of a subject. In many situations that may often be dictated by the condition being treated, active agents achieve improved therapeutic effects when delivered to specific ocular locations. Many ocular tissues, particularly those in the posterior regions of the eye, can be particularly challenging to deliver an active agent into without causing significant damage to the eye. It has now been discovered that active agents can be delivered to such tissues with minimal damage by using a combination of invasive and iontophoretic techniques.

Accordingly, the present invention provides methods for delivering an active agent into the eye of a subject. In one aspect, for example, a method of delivering an active agent into an eye of a subject is provided that includes delivering invasively the active agent into a peripheral tissue of the eye to form a drug reservoir, and applying an electric current to the drug reservoir to thus drive at least a portion of the active agent at least partially through the choroid and deeper into the eye, such as toward a vitreous region. Thus an active agent may be precisely positioned in the tissues along the periphery of the eye using an invasive technique such as injection or implantation, and subsequently delivered further into the eye using an electrical current. This technique avoids significant damage to the choroids, macula, and other sensitive tissues that is seen with other invasive delivery techniques, thus reducing the likelihood of detrimental injury to the eye.

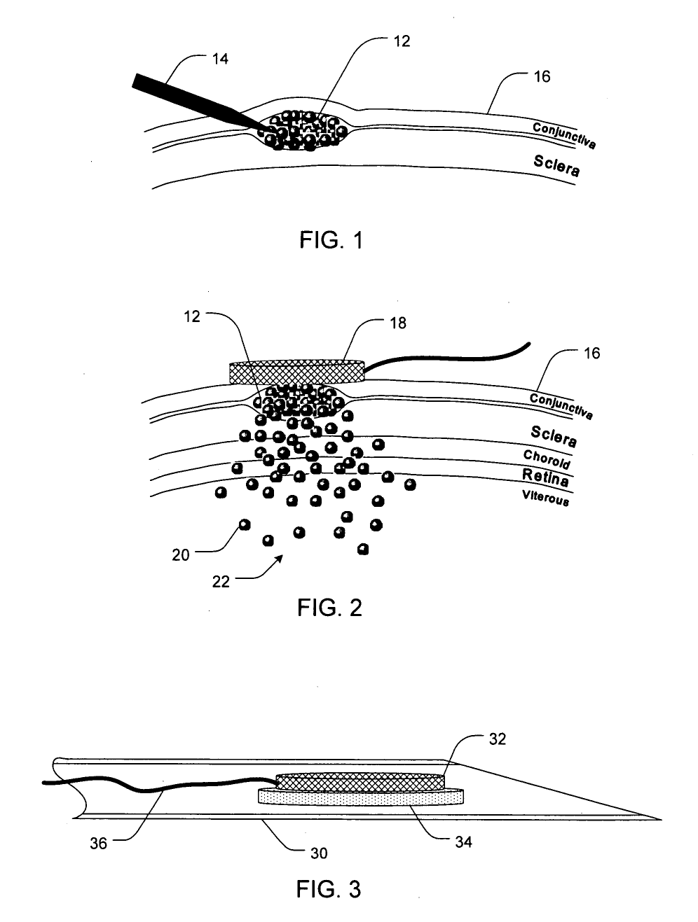

For example, FIG. 1 shows the injection of an active agent through a needle 14 into an eye 16 to form a drug reservoir 12. FIG. 1 shows the drug reservoir being delivered between the conjunctive and the sclera, however any delivery location between the conjunctiva and choroid would be considered to be within the scope of the present invention. For example, in one aspect, the delivery site may be in the suprachoroidal space between the choroid and the sclera.

Invasive ocular delivery may be accomplished by a variety of techniques. For example, in one aspect the active agent may be injected into the eye. Such injections may be accomplished through the use of needles, cannula, etc. In one aspect the injection site may be proximal to the point of entry of the delivery instrument, and thus the drug reservoir is formed near the location where the delivery instrument entered the ocular tissue. In another aspect, the injection site may be remote from the point of entry of the delivery instrument. This would be the case for a cannula that is inserted tangentially and threaded through the ocular tissue to a point that is remote from the initial insertion point. Using such a technique, active agent can be delivered to regions of ocular tissue that would be difficult to reach through conventional injection methods.

In another aspect, the active agent may be implanted into the eye. Such implantation may include hydrogels, liquid reservoirs, polymeric solids or semisolids, etc. In such cases, an incision can be made in the outer ocular tissues, followed by the implantation of the material containing the active agent. It is also considered that implantation may occur through a delivery instrument such as a needle or a cannula. As such, it should be generally considered that there is no limiting distinction between implantation and injection.

A wide range of active agents may be utilized in aspects of the present invention as will be recognized by those of ordinary skill in the art. In fact, any agent that may be beneficial to a subject when administered ocularly may be used. Examples of the active agents that may be used in the treatment of various conditions include, without limitation, analeptic agents, analgesic agents, anesthetic agents, antiasthmatic agents, antiarthritic agents, anticancer agents, anticholinergic agents, anticonvulsant agents, antidepressant agents, antidiabetic agents, antidiarrheal agents, antiemetic agents, antihelminthic agents, antihistamines, antihyperlipidemic agents, antihypertensive agents, anti-infective agents, antiinflammatory agents, antimigraine agents, antineoplastic agents, antiparkinsonism drugs, antipruritic agents, antipsychotic agents, antipyretic agents, antispasmodic agents, antitubercular agents, antiulcer agents, antiviral agents, anxiolytic agents, appetite suppressants, attention deficit disorder and attention deficit hyperactivity disorder drugs, cardiovascular agents including calcium channel blockers, antianginal agents, central nervous system (“CNS”) agents, beta-blockers and antiarrhythmic agents, central nervous system stimulants, diuretics, genetic materials, hormonolytics, hypnotics, hypoglycemic agents, immunosuppressive agents, muscle relaxants, narcotic antagonists, nicotine, nutritional agents, parasympatholytics, peptide drugs, psychostimulants, sedatives, steroids, smoking cessation agents, sympathomimetics, tranquilizers, vasodilators, β-agonists, and tocolytic agents, and mixtures thereof.

Additionally, further examples of active agents may include steroids, aminosteroids, antibacterials, antivirals, antifungals, antiprotozoals, antimetabolites, VEGF inhibitors, ICAM inhibitors, antibodies, protein kinase C inhibitors, chemotherapeutic agents, immunosuppressive agents, neuroprotective agents, analgesic agents, nucleic acid derivatives, aptamers, proteins, enzymes, peptides, polypeptides and mixtures thereof. Specific examples of useful antiviral active agents include acyclovir or derivatives thereof.

Specific examples of active agents may also include hydromorphone, dexamethasone, amikacin, oligonucleotides, F_{ab} peptides, PEG-oligonucleotides, salicylate, tropicamide, methotrexate, 5-fluorouracil, squalamine, triamcinolone acetonide, diclofenac, combretastatin A4, mycophenolate mofetil, mycophenolic acid, bevacizumab (Avastin), ranibizumab (Lucentis), and combinations thereof.

Under a number of circumstances, the active agent used may be a prodrug, or in prodrug form. Prodrugs for nearly any desired active agent will be readily recognized by those of ordinary skill in the art. Additionally, prodrugs with high electromobility which metabolize into drugs with a low aqueous solubility may be beneficial. In this case, an electrically mobile prodrug of a low solubility drug in iontophoresis can be used to create a sustained release system in the eye. Because the prodrug has high electromobility, it is effectively delivered into the eye. The prodrug then converts into the low solubility drug in the eye and the insoluble drug precipitates in the eye. The drug in solid state in the eye will be slowly released into the eye and provide an ocular sustained release condition.

Though any prodrug would be considered to be within the scope of the present invention, examples may include the derivatives of steroids, antibacterials, antivirals, antifungals, antiprotozoals, antimetabolites, VEGF inhibitors, ICAM inhibitors, antibodies, protein kinase C inhibitors, chemotherapeutic agents, immunosuppressive agents, neuroprotective agents, analgesic agents, nucleic acid derivatives, aptamers, proteins, enzymes, peptides, polypeptides, and mixtures thereof. One specific example of a steroid derivative may include triamcinolone acetonide phosphate or other derivatives of triamcinolone acetonide. Another specific example may include dexamethasone phosphate or other derivatives of dexamethasone. As one example of such derivatives, it may be preferable to label a prodrug with one or more phosphate, sulfate, or carbonate functional groups, so the prodrug can be effectively delivered into the eye and form a complex with the precipitating ion.

The active agent being delivered will naturally depend on the condition being treated. The methods of the present invention are particularly well suited for the treatment of ocular diseases and can be utilized as direct, combinatory, and adjunctive therapies due to the relatively high permeability of the eye tissues and the large aqueous compartments in the eye. Examples of eye diseases may include, without limitation, macular edema, age related macular degeneration, anterior, intermediate, and posterior uveitis, HSV retinitis, diabetic retinopathy, bacterial, fungal, or viral endophthalmitis, eye cancers, glioblastomas, glaucoma, and glaucomatous degradation of the optic nerve.

In some aspects of the present invention, a secondary agent may be further delivered to the eye of the subject. In one aspect, the secondary agent may be delivered invasively with the active agent as part of the drug reservoir or merely concomitant therewith. As such, the secondary agent may be delivered by the same delivery instrument as the active agent, or the secondary agent may be delivered by an additional delivery instrument that may function to minimize interaction between the active agent and the secondary agent until after delivery. In another aspect, the secondary agent may be delivered non-invasively through iontophoretic or other means. For example, if the secondary agent has a similar polarity to the active agent, it may be iontophoretically delivered along with the electrical current that is applied to the drug reservoir in order to move the active agent through the choroid. If, on the other hand, the secondary agent has a polarity that is opposite to that of the active agent, the secondary agent may be delivered from the return electrode.

A variety of secondary agents are considered to be beneficial when delivered in conjunction with the active agent. It should be understood that any secondary agent that provides a therapeutic effect in addition to that of the active agent, or that benefits the delivery or action of the active agent, should be considered to be with in the scope of the present invention. Non-limiting examples of such secondary agents may include depot forming agents, active agents, vasoconstrictor agents, solubility modifying agents, and combinations thereof.

In some aspects, a secondary agent may be utilized to reduce the in-vivo movement/clearance of the active agent in the eye. It is contemplated that various means for restricting or slowing such movement may improve the effectiveness of the active agent therapy. In one aspect, the in-vivo movement may be restricted by constriction of the blood vessels exiting an area in which the active agent is being delivered or precipitated. Such constriction may be induced by the administration of a secondary agent such as a vasoconstrictor agent. Specific non-limiting examples of vasoconstrictor agents may include α-agonists such as naphazoline, and tetrahydrozoline, sympathomimetics such as phenylethylamine, epinephrine, norepinephrine, dopamine, dobutamine, colterol, ethylnorepinephrine, isoproterenol, isoetharine, metaproterenol, terbutaline, metearaminol, phenylephrine, tyramine, hydroxyamphetamine, ritrodrine, prenalterol, methoxyamine, oxymetazoline, albuterol, amphetamine, methamphetamine, benzphetamine, ephedrine, phenylpropanolamine, methentermine, phentermine, fenfluramine, propylhexedrine, diethylpropion, phenmetrazine, and phendimetrazine. In one specific aspect, the vasoconstrictor agent may include oxymetazoline. Vasoconstrictor agents can be administered either before or concurrently with the administration of the active agent. Though administration of the vasoconstrictor may occur following administration of the active agent, the results may be less effective than prior or concurrent administration. Additionally, in some aspects, the vasoconstrictor agent may have the same polarity as the active agent and administered concurrently with the active agent. Similarly, the vasoconstrictor agent may have the opposite polarity as active agent, and thus be administered from a return electrode.

Although certain active agents form drug depots in the eye through interaction with endogenous materials, it is contemplated that depot forming agents may be delivered as secondary agents to cause or improve the formation of a drug depot. In situations where a depot forming agent is delivered along with the active agent, it may be beneficial to preclude interaction between the active agent and the depot forming agent until both compounds are present within the eye at a location suitable for drug depot formation. In some aspects, the drug depot may be formed at the injection site. In other aspects, it may be beneficial to cause formation of the drug depot to occur remote from the injection site following iontophoretic delivery of the active agent through the choroid. This situation may be particularly beneficial for drug depots that may not exhibit substantial movement in response to an electric field. Although numerous methods of forming a drug depot remote from the injection site are contemplated, in one aspect the depot forming agent may be delivered invasively or non-invasively from a site that is remote from the injection site of the active agent. In such a situation, the spacing of the active agent and depot forming agent delivery sites may be such that the iontophoretic current moves both agents into contact at the desired location.

The in-vivo reaction between the active agent and the depot forming agent will cause the active agent or a derivative thereof to form a depot. In one aspect such a depot forming mechanism may be a change in the solubility of the active agent or a derivative of the active agent, thus causing precipitation and subsequent depot formation. This depot of active agent complex is then able to deliver a therapeutic compound to the biological system over time, particularly for those depots formed remote from the injection site. In some aspects, the depot forming agent may not react directly with the active agent, but still function to facilitate the formation of a sustained release depot. In such a case, the depot forming agent may react with an area of a local environment to cause an alteration therein, and the active agent would then react with the altered area of the local environment to form a depot as a result of the changes facilitated by the depot forming agent. Further details on such depot administration and depot agents can be found in U.S. patent application Ser. Nos. 11/238,144 and 11/238,104, both filed on Sep. 27, 2005, both of which are incorporated herein by reference.

Various reactions are contemplated that result in a sustained release depot being formed. The reaction between the active agent and the depot forming agent may include an ionic association. Accordingly, in one aspect the depot forming agent can have at least one opposite charge to at least one of the charged groups on the active agent. In another aspect, the depot forming agent can have more than one charge and will be capable of being juxtaposed with more than one charge on the active agent. In yet another aspect, the charges on the depot forming agent can be polyvalent, allowing more than one active agent ion to enter the depot complex. This allows stronger associations between complexing depot forming agents, thereby lowering the solubility constant of the depot complex, Ksp, thus increasing the duration of therapy. In one aspect, the depot forming agent may be an ion. Examples of useful depot forming agents include without limitation, Ca²⁺, Sn²⁺, Fe²⁺, Fe³⁺ Mn²⁺, Mg²⁺, Zn²⁺, NH₄⁺, ions of the transition metals in the periodic tables, PO₄³⁻, CO₃²⁻, SO₄²⁻, organic cations, organic anions, polyvalent metals, chelation agents, and ionic pharmaceutical excipients generally used in the pharmaceutical industry or known to the people skilled in the art. The depot forming agents preferably have more than one charge for effective iontophoretic delivery and for effectively precipitating the active agent. In one aspect, the depot forming agent may have an adequate ionic charge for both effective iontophoretic delivery and effectively reacting with the active agent to form the sustained release depot.

The ratio of depot forming agent to active agent could be one to one. However, in the case of polyvalent depot forming agents, more than one active agent may complex with the same depot forming agent to form a depot complex. In one aspect, the depot complex may have a ratio of depot forming agent to active agent of from about 1:1 to about 1:4. In another aspect, the ratio may be about 1:1. In a further aspect, the ratio may be about 1:2. In yet another aspect, the ratio may be about 1:3. In yet a further aspect, the ratio may be about 1:4. In one more aspect, the ratio of depot forming agent to active agent may be from about 4:1 to about 1:4.

Two or more depot forming agents can be used at the same time to form the sustained release depot. With multiple depot forming agents, the concentration of each depot forming agent for precipitating the same total amount of active agent in the eye can be reduced. This effectively reduces the concentrations of the depot forming agent in the eye during and after delivery, so the depot forming agent concentrations are always below the levels that may cause adverse effects in the eye. The use of multiple depot forming agents also provides other advantages. For example, sustained release can be further controlled by using multiple depot forming agents that have different depot complex-Ksp values.

Other examples of depot forming agents may include, without limitation, catalysts, polymerization initiators, pegylating agents, solvents, pH, thermal, or ionic strength sensitive polymers, active agents used in the treatment of eye diseases, aminosteroids such as squalamine, and combinations and mixtures thereof.

As has been described, in one aspect an endogenous depot forming agent may facilitate the creation of a depot upon administration of the active agent. Examples of such agents may include without limitation, various enzymes, ascorbate, lactate, citrate, various amino acids, calcium, magnesium, zinc, iron, chloride, fluoride, as well as ions found in the tissues and vitreous of the eye. In such cases, the presence of such a substance inside the body may be relied upon in order to form the depot and once the active agent has been delivered. Alternatively, such substances may be delivered to the body if they are not thought to be present in sufficient concentration to form a depot.

In one aspect, for example, an electrically mobile prodrug of a low solubility active agent, as is the case with triamcinolone acetonide and triamcinolone acetonide phosphate, can be used to create a sustained release system in the eye. Because the triamcinolone acetonide phosphate prodrug has high electromobility, it is effectively delivered into the eye. The prodrug then converts into the lower solubility triamcinolone acetonide in the eye and the lower solubility drug precipitates. The active agent in solid state in the eye will be slowly released into the eye and provide an ocular sustained release condition.

Following delivery of the active agent into the suprachoroidal space to form a drug reservoir, an electric current may be applied to the drug reservoir to thus drive at least a portion of the active agent at least partially through the choroid and deeper into the eye. In one aspect, FIG. 2 shows an electrode 18 positioned non-invasively over the drug reservoir 12. Application of an electrical current through the drug reservoir 12 from the electrode 18 drives at least a portion of the active agent 20 deeper into the eye, such as into the vitreous region.

In another aspect, the electrode may be invasively inserted into the eye, either along with the drug reservoir or independently therefrom. In one aspect, FIG. 3 shows a hypodermic needle 30 containing an electrode 32 coupled to a drug reservoir in the form of a sponge 34. Conductive leads 36 from the electrode 32 are fed through the interior of the needle 30, and are configured to coupled to a current-generating device. The needle 30 is inserted into the ocular tissue and the electrode and reservoir are ejected therefrom and positioned appropriately for the delivery of the active agent. Such an insertion may also be accomplished with a cannula or other delivery instrument.

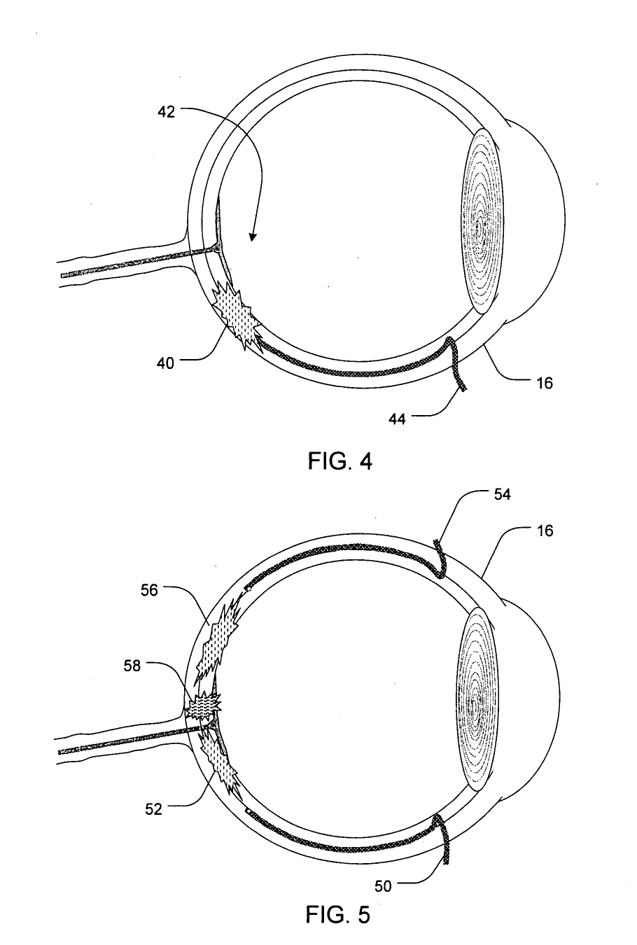

FIG. 4 shows one aspect in which a drug reservoir 40 is delivered and iontophoretically driven deeper into ocular tissues in the posterior regions 42 of the eye 16. A cannula 44 containing an electrode and a drug reservoir (not shown) are inserted into peripheral tissues of the eye and threaded back to a more posterior position. Following placement of the cannula 44, an active agent is delivered, followed by an electrical current provided by the electrode in the cannula 44. Such electrical current drives the active agent deeper into the eye than would be possible with active agent delivery alone.

FIG. 5 shows an aspect whereby a depot forming agent is delivered along with the active agent. In such an aspect, an active agent cannula 50 having an electrode and a reservoir containing an active agent is positioned in a posterior region of the eye 16 as was described in FIG. 4. Electrical current applied through the active agent cannula 50 will thus deliver active agent as an active agent reservoir 52 into the surrounding tissue or space between tissues. A secondary cannula 54 is inserted and positioned in a similar manner as the active agent cannula 50. The secondary cannula 54 may contain a depot forming agent in a reservoir and an electrode. The depot forming agent may be delivered to form a depot forming agent reservoir 56. Depending on the distance between the active agent reservoir 52 and the depot forming agent reservoir 56, further electrical current may be applied across both reservoirs to drive the agents together, thus causing them to react with one another and to form a drug depot 58. In an alternative embodiment, the active agent and the depot forming agent may be delivered to form respective reservoirs in the peripheral tissues without the application of electrical current. As such, a drug depot may be formed as the active agent and the depot forming agent move together and interact via diffusion.

The electrodes of the present invention are designed to deliver electrical current across a drug reservoir to iontophoretically deliver the active agent located therein. The electrodes can be of any material or manufacture known to one skilled in the art. Various examples include metal electrodes, conductive glass electrodes, etc. A single electrode may be coupled to a single reservoir or to multiple reservoirs depending on the particular configuration of a given electrode assembly. Additionally, in some aspects of the present invention, an electrode may also be a reservoir, with the depot forming agent being delivered from the body of the electrode.

A return electrode is utilized to complete an electric circuit with the active agent electrode. The return electrode may be located invasively within the eye, on the surface of the eye, or remote from the eye on, for example, an earlobe or eyelid. However, placing the return electrode either invasively within the eye, or on the surface of the eye may facilitate the passage of electrical current transsclerally into the eye under the active agent electrode, particularly when current movement across the surface of the eye is limited.

The present invention also provides techniques for forming a drug depot in the eye in which the use of an iontophoretic current is optional. Such techniques may be useful for targeting difficult to reach portions of the eye, such as at or near the posterior pole. For example, in one aspect a drug reservoir may be invasively delivered to a first delivery site in an area of peripheral tissue. A depot forming agent reservoir may be invasively delivered to a second delivery site in an area of peripheral tissue that is distinct from the first site. The first and second delivery sites may be respectively located such that a drug depot is formed between the two sites as a result of the movement and subsequent interaction of the active agent and the depot forming agent. This technique may allow the delivery of an active agent and a depot forming agent to separate delivery sites in peripheral tissues of the eye, followed by subsequent movement and interaction of these agents to form a drug depot at a more posterior position in the eye. The movement of the active agent and/or the depot forming agent may be a result of diffusion, exerted pressure from the delivery instrument, or any other technique known. By varying the relative location of the delivery sites, a drug depot may be formed in locations of the eye that may be very difficult and/or potentially dangerous to inject to by traditional methods.

In addition to the relative spatial locations of the delivery site, the location of formation of the drug depot may be further varied through the timing of the delivery of the active agent compared to the depot forming agent. For example, a depot forming agent that has been delivered prior to the active agent may diffuse further away from the delivery site, thus affecting the location where the active agent and the depot forming agent come into contact. As such, in one aspect the depot forming agent may be delivered simultaneously with the active agent. In another aspect, the depot forming agent may be delivered prior to delivery of the active agent. In yet another aspect, the depot forming agent may be delivered following delivery of the active agent. Additionally, it should be noted that these aspects should not be limited to the use of active agents in combination with depot forming agents, but may also be applicable to active agents in combination with other secondary agents.

It should be understood that the above-described arrangements are only illustrative of the application of the principles of the present invention. Numerous modifications and alternative arrangements may be devised by those skilled in the art without departing from the spirit and scope of the present invention. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method of delivering an active agent into an eye of a subject, comprising:
delivering invasively an active agent into a peripheral tissue of the eye to form a drug reservoir; and
applying an electric current to the drug reservoir to thus drive at least a portion of the active agent at least partially through the choroid.

2. The method of claim 1, wherein the electric current is applied to the drug reservoir from a non-invasively positioned electrode.

3. The method of claim 2, further comprising completing an electrical circuit with the non-invasive electrode via a non-invasive return electrode positioned on an eye surface.

4. The method of claim 1, wherein the electric current is applied to the drug reservoir from an invasively positioned electrode.

5. The method of claim 4, wherein the invasive electrode is located within the peripheral tissue.

6. The method of claim 4, wherein delivering the active agent further includes implanting an invasive electrode having an associated drug reservoir containing the active agent into the peripheral tissue.

7. The method of claim 4, further comprising completing an electrical circuit with the invasive electrode via a return electrode positioned within the peripheral tissue.

8. The method of claim 7, further comprising iontophoretically delivering a secondary agent from a secondary reservoir associated with the return electrode into the eye of the subject.

9. The method of claim 8, wherein the secondary agent is a depot forming agent.

10. The method of claim 1, further comprising allowing the active agent to diffuse along the peripheral tissue prior to applying the electrical current.

11. The method of claim 1, wherein the active agent is selected from the group consisting of hydromorphone, dexamethasone, dexamethasone phosphate, amikacin, oligonucleotides, F_{ab} peptides, PEG-oligonucleotides, salicylate, tropicamide, methotrexate, 5-fluorouracil, squalamine, triamcinolone acetonide, triamcinolone acetonide phosphate, diclofenac, combretastatin A4, mycophenolate mofetil, mycophenolic acid, bevacizumab, ranibizumab, and prodrugs and combinations thereof.

12. The method of claim 11, wherein the active agent is triamcinolone acetonide phosphate.

13. The method of claim 11, wherein the active agent is dexamethasone phosphate.

14. The method of claim 1, further comprising delivering a secondary agent to the eye of the subject.

15. The method of claim 14, wherein the secondary agent is invasively delivered with the drug reservoir.

16. The method of claim 14, wherein the secondary agent is non-invasively delivered with the electric current.

17. The method of claim 14, wherein the secondary agent is a member selected from the group consisting of depot forming agents, active agents, vasoconstrictor agents, solubility modifying agents, and combinations thereof.

18. The method of claim 14, wherein the secondary agent is a vasoconstrictor agent.

19. The method of claim 18, wherein the vasoconstrictor agent is a member selected from the group consisting of naphazoline, tetrahydrozoline, phenylethylamine, epinephrine, norepinephrine, dopamine, dobutamine, colterol, ethylnorepinephrine, isoproterenol, isoetharine, metaproterenol, terbutaline, metearaminol, phenylephrine, tyramine, hydroxyamphetamine, ritrodrine, prenalterol, methoxyamine, oxymetazoline, albuterol, amphetamine, methamphetamine, benzphetamine, ephedrine, phenylpropanolamine, methentermine, phentermine, fenfluramine, propylhexedrine, diethylpropion, phenmetrazine, phendimetrazine, and combinations thereof.

20. The method of claim 19, wherein the vasoconstrictor agent is oxymetazoline.

21. The method of claim 17, wherein the secondary agent is a depot forming agent.

22. A method of forming a sustained release drug depot at or near a posterior pole of an eye of a subject, comprising:
delivering invasively an active agent into a peripheral tissue of the eye to form a drug reservoir at a first delivery site;
delivering a depot forming agent into a peripheral tissue of the eye to form a depot forming agent reservoir at a second delivery site, wherein the first delivery site and the second delivery site are spatially distinct; and
allowing the active agent and the depot forming agent to diffuse to an intermediate location between the first site and the second site to form a drug depot.

23. The method of claim 22, wherein the active agent is water soluble.

24. The method of claim 22, wherein the intermediate location is in the posterior pole of the eye.
